(19) United States
(12) Patent Application Publication
(10) Pub. No.: US 2021/0158970 A1
(43) Pub. Date: May 27, 2021

(54) DISEASE SIMULATION AND IDENTIFICATION IN MEDICAL IMAGES

(71) Applicant: International Business Machines Corporation, Armonk, NY (US)

(72) Inventors: Benedikt Graf, Charlestown, MA (US); Arkadiusz Sitek, Ashland, MA (US); Yiting Xie, Cambridge, MA (US); AMIN KATOUZIAN, Lexington, MA (US); PEDRO LUIS ESQUINAS FERNANDEZ, Etobicoke (CA); Lilla Boroczky, Mount Kisco, NY (US); Mark D. Bronkalla, Waukesha, WI (US)

(21) Appl. No.: 16/691,824

(22) Filed: Nov. 22, 2019

(51) Int. Cl. G16H 50/50; G16H 30/40; G16H 30/20; G16H 50/20; G06N 3/08; G06T 11/60; G06T 7/00

(52) U.S. Cl. CPC G16H 50/50; G16H 30/40; G16H 30/20; G16H 50/20; G06T 2207/20084; G06T 11/60; G06T 7/0012; G06T 2210/41; G06T 2207/20221; G06N 3/08

(57) ABSTRACT

A method, system, and computer program product for disease simulation and identification in medical images. The method generates a set of synthetic projection images from first medical imaging data of a first imaging type. Second medical imaging data of a second imaging type is projected onto the set of synthetic projection images. One or more synthetic images of the set of synthetic projection images are modified to generate a set of modified projection images based on the second medical imaging data. The method generates an imaging model based on the set of modified projection images. The method obtains a patient medical image of the first imaging type and identifies an attribute of interest on the patient medical image based on the imaging model.

### BACKGROUND

Neural networking algorithms are employed in many areas of data analysis. Neural networking and deep learning algorithms have been used in image recognition. Such algorithms employ large data sets for training of neural networking models used to perform data analysis, such as image recognition. The data sets are often labeled to designate attributes of interest within individual or groups of discrete files within the data sets. The size and quality of data sets used for training, tuning, and testing of neural networking algorithms often materially affect the quality and precision of the neural networking models.

### SUMMARY

According to an embodiment described herein, a computer-implemented method for disease simulation and identification in medical images is provided. The method generates a set of synthetic projection images from first medical imaging data of a first imaging type. Second medical imaging data of a second imaging type is projected onto the set of synthetic projection images. At least a portion of the set of synthetic projection images are modified to generate a set of modified projection images based on the second medical imaging data. The method generates an imaging model based on the set of modified projection images. A patient medical image of the first imaging type is obtained. The method identifies an attribute of interest on the patient medical image based on the imaging model.

According to an embodiment described herein, a computer-implemented method for disease simulation and identification retrieves second medical imaging data of a second imaging type, where a set of synthetic projection images is a first set of synthetic projection images. The method generates a second set of synthetic projection images from the second medical imaging data. The second set of synthetic projection images have the second imaging type. According to an embodiment described herein, a second imaging model is generated based on the second set of synthetic projection images and the second medical imaging data is projected onto the first set of synthetic projection images based on the second imaging model. According to embodiments described herein, a system and a computer program product are described which are similar to or the same as the above-referenced computer-implemented method.

According to an embodiment described herein, a computer-implemented method for disease simulation and identification identifies an area of interest within a patient medical image. The area of interest indicates an area of the patient depicted in a patient medical image. The computer-implemented method selects an area model based on the area of interest. The area model is an imaging model generated for a specified bodily area associated with the area of interest. The area model facilitates identification of diseases or irregularities within the area of interest. According to embodiments described herein, a system and a computer program product are described which are similar to or the same as the above-referenced computer-implemented method.

According to an embodiment described herein, a system for disease simulation and identification in medical images is provided. The system includes one or more processors and a computer-readable storage medium, coupled to the one or more processors, storing program instructions that, when executed by the one or more processors, cause the one or more processors to perform operations. The operations generate a set of synthetic projection images from first medical imaging data of a first imaging type. Second medical imaging data of a second imaging type is projected onto the set of synthetic projection images. At least a portion of the set of synthetic projection images are modified to generate a set of modified projection images based on the second medical imaging data. The operations generate an imaging model based on the set of modified projection images. A patient medical image of the first imaging type is obtained. The operations identify an attribute of interest on the patient medical image based on the imaging model.

According to an embodiment described herein a computer program product for disease simulation and identification in medical images is provided. The computer program product includes a computer readable storage medium having program instructions embodied therewith, the program instructions being executable by one or more processors to cause the one or more processors to perform operations including generating a set of synthetic projection images from first medical imaging data of a first imaging type. Second medical imaging data of a second imaging type is projected onto the set of synthetic projection images. At least a portion of the set of synthetic projection images are modified to generate a set of modified projection images based on the second medical imaging data. The computer program product generates an imaging model based on the set of modified projection images. A patient medical image of the first imaging type is obtained. The computer program product identifies an attribute of interest on the patient medical image based on the imaging model.

### BRIEF DESCRIPTION OF THE DRAWINGS

FIG. 1 depicts a block diagram of a computing environment for implementing concepts and computer-based methods, according to at least one embodiment.

FIG. 2 depicts a flow diagram of a computer-implemented method for disease simulation and identification in medical images, according to at least one embodiment.

FIG. 3 depicts a flow diagram of a computer-implemented method for disease simulation and identification in medical images, according to at least one embodiment.

FIG. 4 depicts a block diagram of a computing system for disease simulation and identification in medical images, according to at least one embodiment.

FIG. 5 is a schematic diagram of a cloud computing environment in which concepts of the present disclosure may be implemented, in accordance with an embodiment of the present disclosure.

FIG. 6 is a diagram of model layers of a cloud computing environment in which concepts of the present disclosure may be implemented, in accordance with an embodiment of the present disclosure.

### DETAILED DESCRIPTION

The present disclosure relates generally to methods for disease simulation and identification in medical images. More particularly, but not exclusively, embodiments of the present disclosure relate to a computer-implemented method for generating synthesized medical images to train imaging models configured to identify abnormalities within patient medical images. The present disclosure relates further to a related system for disease simulation and identification in medical images, and a computer program product for operating such a system.

Computer learning (e.g., neural networking and deep learning algorithms) is used across various applications for analyzing data sets. Computer learning systems may generate models from large and organized data sets. In some industries and applications, sufficiently large data sets have been collected for training varying types or aspects of neural networking models. Collecting large data sets in the medical imaging domain presents challenges distinct from other applications or fields. A prevalence of some diseases may be low, precluding collection of large and varied data sets relating to those diseases. Additionally, diseases may present with a wide variability. The variability of disease may be attributed to timing of a diagnosis or imaging of the disease, peculiarities of patients having the disease, location variation of diseases, and differences in severity of a specified disease. Similarly, technical aspects may affect the variability in imaging diseases. These technical factors may include patient position, scanner or imaging model, image acquisition and reconstruction parameters, combinations thereof, and other pertinent factors. As such, training neural networking models on data sets with sufficient variability may present significant challenges.

As an example, pneumothorax is a condition which consists of air leaking into the pleural space of a chest cavity. This condition may be detected using chest x-rays. The prevalence of pneumothorax in chest x-rays is typically one to three percent. The appearance of the pneumothorax on x-ray images varies significantly. The variance may be attributable to a size of the pneumothorax, ranging from small pockets of air to a total collapse of a lung with a large volume in a chest cavity occupied by air. Further, variability may occur due to patient positioning. For example, intrathorax air pockets may accumulate in different parts of the chest cavity, based on a patient's position. The positioning may make the air pockets appear differently on x-rays. Additionally, image quality of x-rays may vary. A wide range of image quality for chest x-rays may be experienced due to variations in positioning of the x-ray source, an energy spectrum of the source, particularities of a patient, and detectors used. Thus, building a data set of pneumothorax images may present difficulties in shared attributes suitable for modeling and identification in later patient images.

By way of further example, differences in patient and detector positioning introduce acquisition variabilities to medical imaging. In cases where a detector and patient are not squarely aligned may result in blurring, structure distortion, or non-standard structure overlap such as in the mediastinum. Patient positioning errors may result in medical images being rotated, clipped, or otherwise distorted. Portable imaging devices, such as portable or upright chest x-rays, may be taken with a patient in a sitting position facing away from a detector. Portable imaging devices may take supine images (e.g., patients laying on a table or bed) but may generate poorer quality images due the variabilities of placement and orientation of the imaging device and patient within a non-standard imaging room. Each of these images, errors, or variances may be simulated and adjusted for, by embodiments of the present disclosure, for accurate identification of attributes of interest in patient medical images.

Synthetic images can also be generated without simulating disease or abnormalities. In this case, the three-dimensional images can provide more information about the extent of the abnormality or disease than a two-dimensional projection image. The information from the three-dimensional images can be used as a stronger ground truth for the training of the models with the synthetic projection images and enable more accurate identification of attributes of interest in patient medical images. In some instances, models are trained using combinations of simulated or synthetic images with abnormalities and without abnormalities.

Embodiments of the present disclosure enable collection of significantly large data sets for model generation by using medical images as a starting point. Synthetic images are generated from medical imaging data. Synthetic images can include computer-generated visual representations of medical abnormalities that are based on medical imaging data. Medical abnormalities in the synthetic images may represent diseases; tissue damage; obstructions; image distortion from patient, detector, or emitter positioning or orientation; resolution issues; noise issues or ratios; emitter or detector sensitivity; tissue density or absorption issues; combinations thereof; or any other suitable and relevant abnormalities in medical imaging. Embodiments of the present disclosure enable simulation of diseases and abnormalities in medical images to generate training samples to improve training of image analytics and image recognition algorithms and models. The synthetic images may be generated by introducing abnormalities of interest using image processing operations including segmenting and reshaping portions of images, introducing foreign objects, introducing voids, combinations thereof, and any other suitable modification of medical images. Embodiments of the present disclosure enable reductions in time, expense, and computing cycles in collecting medical images and generating models. Further, embodiments of the present disclosure enhance accuracy and precision of imaging models in detecting abnormalities within patient medical images.

Some embodiments of the concepts described herein may take the form of a system or a computer program product. For example, a computer program product may store program instructions that, when executed by one or more processors of a computing system, cause the computing system to perform operations described above with respect to the computer implemented method. By way of further example, the system may comprise components, such as processors and computer readable storage media. The computer readable storage media may interact with other components of the system to cause the system to execute program instructions comprising operations of the computer implemented method, described herein. For the purpose of this description, a computer-usable or computer-readable medium may be any apparatus that may contain means for storing, communicating, propagating, or transporting the program for use, by, or in connection with, the instruction execution system, apparatus, or device.

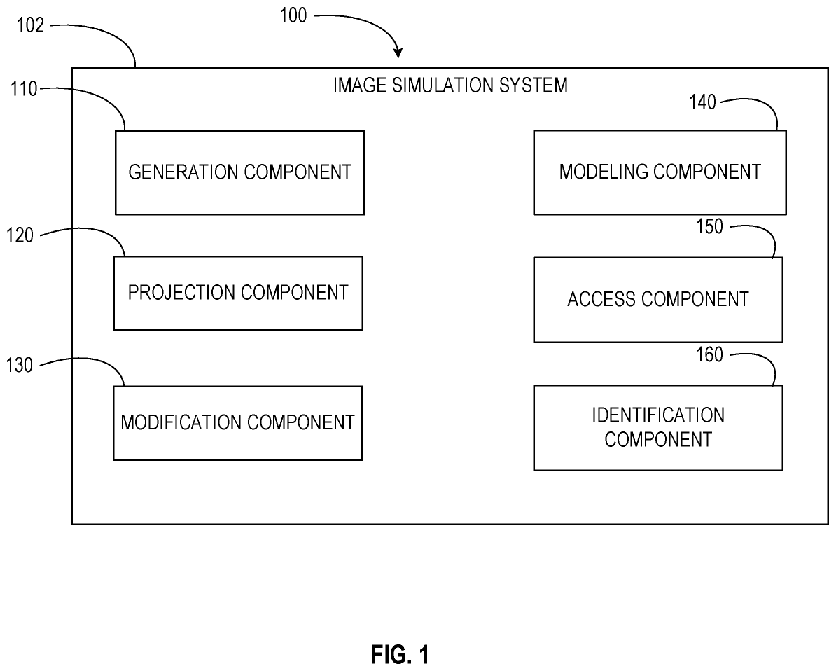

Referring now to FIG. 1, a block diagram of an example computing environment 100 is shown. The present disclosure may be implemented within the example computing environment 100. In some embodiments, the computing environment 100 may be included within or embodied by a computer system, described below. The computing environment 100 may include an image simulation system 102. The image simulation system 102 may comprise a generation component 110, a projection component 120, a modification component 130, a modeling component 140, an access component 150, and an identification component 160. The generation component 110 generates synthetic projection images from medical imaging data of varying imaging types. The projection component 120 projects medical imaging data of a different imaging type onto synthetic projection images. The modification component 130 modifies synthetic projection images to create modified projection images based on projected medical imaging data. The modeling component 140 generates imaging models from synthetic projection images and modified projection images. The access component 150 accesses or obtains patient medical images of specified imaging types. The identification component 160 identifies attributes of interest on patient medical images based on one or more imaging models. Although described with distinct components, it should be understood that, in at least some embodiments, components may be combined or divided, or additional components may be added, without departing from the scope of the present disclosure.

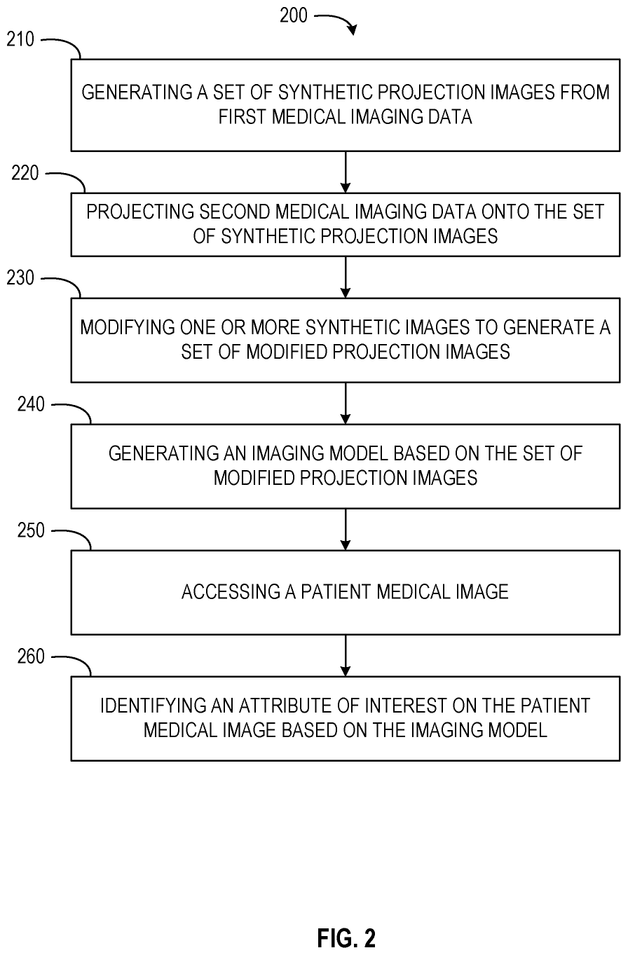

Referring now to FIG. 2, a flow diagram of a computer-implemented method 200 is shown. The computer-implemented method 200 is a method for disease simulation and identification in medical images. In some embodiments, the computer-implemented method 200 may be performed by one or more components of the computing environment 100, as described in more detail below.

At operation 210, the generation component 110 generates a set of synthetic projection images from first medical imaging data. The first medical imaging data may be understood as a source image or source images. Each source image or source image set may correspond to a separate set of synthetic images. Each source image or set of source images may have one or more elements or attributes inserted as part of the process for generating synthetic images. Multiple variations of each source image or set of source images may be generated as an individual set of synthetic images.

In some embodiments, the first medical imaging data includes imaging data of a first imaging type. For example, the first imaging type may be two-dimensional x-ray images. The generation component 110 may generate the set of synthetic projection images to any suitable size. In some embodiments, the set of synthetic projection images may be generated as a large enough data set to provide a plurality of sets of synthetic projection images. A portion of the set of synthetic projection images may be used in varying aspects of model training.

In some embodiments, the generation component 110 generates a plurality of related synthetic projection images from the first medical imaging data. The generation component 110 may generate synthetic projection images having specified attributes, abnormalities, or diseases. The attributes may include aortic aneurysms, solid tumors, lung nodules, lung diseases, lesions (e.g., breast lesions and liver lesions), pneumothorax, pulmonary embolisms, coronary vessel abnormalities, ventricular abnormalities, fractures (e.g., bone fractures), calcifications, atheromatous plaques, combinations thereof, and other suitable and relevant abnormalities. Attributes may also include imaging abnormalities, such as patient positioning errors, detector positioning errors, detector/emitter positioning or orientation errors, detector/emitter distance errors, and other imaging errors or irregularities. Further, attributes may include tissue density or absorption abnormalities. The generation component 110 may randomly select imaging attributes or select imaging attributes on a stepwise basis. In such embodiments, the generation component 110 may generate large batches of simulated medical images with varied imaging attributes from a single source image. In some instances, the imaging attributes selected or applied to each resulting simulated image or batch of simulated images may be tracked and stored in association with the simulated images. The tracked and labeled imaging attributes may act as labeling for model training, generation, tuning, and testing.

When generating projection images for aortic aneurysms, the generation component 110 may generate segmentation in an aorta on a CT scan using image interpolation. The segmentation may increase or decrease a diameter of the aorta at a specified location to introduce a bulge in the aorta. The generation component 110 may perform similar operations on x-ray images. In generating lung nodules, the generation component 110 may identify one or more healthy locations in a lung on a CT scan or x-ray. The generation component 110 may use image segmentation and a generative adversarial network (GAN) to modify portions of the healthy areas to add one or more lung nodules. In simulating other lung diseases, the generation component 110 may add nodule variants, such as solid nodules, semi-solid nodules, and ground-glass opacities, at locations of healthy area, attachments to vasculatures, lung walls, or peripheries. In generating lesion projection images, the generation component 110 may modify x-ray or magnetic resonance images to generate breast tomosynthesis, lesions on a liver, or any other suitable areas of a body depicted in an image. Pneumothorax may be generated by image segmentation of a lung on a CT scan to generate voids, simulate collapse of a lung, or other related conditions. The generation component 110 may realign portions of coronary vessels and change an appearance of a cardiac cycle or introduce wall motion or valve disorder irregularities to generate projection images relating to coronary functions. Fractures may be generated using image segmentation on CT scans or x-rays. The generation component 110 may also incorporate GAN to simulate a presence of non-displaced fractures. Simulating intermediate images, between medical images of healthy tissues and medical images of identified abnormalities or attributes of interest, the synthetic projection images may enable or aid in fusion and registration of pre-operative and intra-operative images.

In some embodiments, attributes include imaging abnormalities, unrelated to diseases. Imaging abnormalities may include patient positioning errors, detector positioning errors, detector/emitter positioning or orientation errors, detector/emitter distance errors, and other imaging errors or irregularities. Additionally, abnormalities may include tissue density and absorption abnormalities. In some instances, the imaging attributes selected or applied to each resulting simulated image or batch of simulated images may be tracked and stored in association with the simulated images. The tracked and labeled imaging attributes may act as labeling for model training, generation, tuning, and testing. Where the imaging attributes include tissue absorption or tissue density, the generation component 110 may generate imaging attributes using ray trace emulation. For example, tissue absorption modeling, as used in NM SPECT and PET for correction of radio tracer photons from CT images, may be used to emulate different x-ray beam hardness.

In some embodiments, the attributes may correspond to selected synthetic images, sets of synthetic images, or batches of synthetic images. In some instances, the attributes are selected for each image, set of images, or batch of images. The attributes may also be automatically selected by the generation component 110. The attributes may also be randomly selected or selected in a stepwise manner. The selection, random selection, or stepwise selection of attributes for inclusion in the set of synthetic images may progressively generate a training set of synthetic images for use in generating diagnostic models, as discussed in more detail below. In some instances, the generation component 110 identifies imaging attributes within each synthetic image or in an associated data structure. Identified attributes, associated with each image, may be used as labels to facilitate model training, tuning, and testing.

In some embodiments, the generation component 110 performs batch operations, automating the generation of sets of synthetic images. The batched generation of synthetic images may be based on a single source image. The batched generation of synthetic images may also be based on a set or corpus of source images (e.g., hundreds or thousands of images), attributes or attribute sets for each batch, and parameter sets generated or selected for each batch. The sets of synthetic images may be automatically generated. Parameter sets may enable multiple variations for each source image or source image set to generate one or more simulated output images. As discussed herein, the embodiments of the present disclosure may enable automatic or semi-automatic generation of any number of synthetic images. For example, the present disclosure may generate a single synthetic image, hundreds of synthetic images, or thousands of synthetic images with sets of synthetic images including one or more of the generated images.

At operation 220, the projection component 120 projects second medical imaging data onto the set of synthetic projection images. In some embodiments, the second medical imaging data includes data of a second imaging type. For example, the second medical imaging data may have an imaging type of volumetric computerized tomography (CT) scan data. The CT volume data may be projected onto the set of synthetic projection images (e.g., synthetic x-ray images). The projection component 120 may project the second medical imaging data using forward projection operations. In some embodiments, the projection component 120 converts the second medical imaging data from the second imaging type to the first imaging type, then projecting the second medical imaging data onto the set of synthetic projection images.

At operation 230, the modification component 130 modifies one or more synthetic images of the set of synthetic projection images. In some embodiments, modification of the synthetic images generates a set of modified projection images based on the second medical imaging data. For example, the set of synthetic projection images, initially generated as two-dimensional images, may be rendered as three-dimensional models by the modification component 130. The modification component 130 may use a graphics processing unit to render the set of synthetic projection images as the set of modified projection images.

In some embodiments, the modification component 130 modifies the one or more synthetic images by augmenting at least a portion of the set of synthetic projection images with a set of image tracking points. The set of image tracking points may aid in correlating aspects or attributes of two-dimensional synthetic images to source images that are three-dimensional. In some embodiments, augmenting the set of synthetic projection images with image tracking points enables visualization of the synthetic images in varying user interfaces, allowing manipulation of the synthetic projection images in varying computing environments. The modification component 130 may augment the set of synthetic projection images by adding virtual reality, augmented reality, virtual cubes or exclusion cubes, or any other suitable tracking points. The virtual cubes or exclusion cubes may be rotatable along an axis.

In some embodiments, operations 210, 220, and 230 may be performed contemporaneously or in a single image generation operation. In some embodiments, components of the image simulation system 102 obtain a source image (e.g., the first medical imaging data of the first imaging type), identify a second imaging type to be generated from the source imaging data, model a second medical imaging data of the second imaging type from the first imaging data, and generate the set of synthetic images of the second imaging type.

The image simulation system 102 may identify a parameter set for the second imaging type, to aid in modeling and generation of the synthetic projection images. The parameter set are physical properties or aspects of one or more of a target imaging device, a patient position, a detector, or the second imaging type. The parameter set may include a sensitivity, a resolution, a noise, a noise ratio, x-ray source to detector geometry (e.g., a source to image distance), orientation of the patient image volume within the x-ray geometry (affecting rotations and geometric magnification of the structures), x-ray beam attenuation characteristics, combinations thereof, or any other suitable and relevant physical properties or parameters. In some embodiments, selection and adjustment of the parameter set may correct for, manipulate, or simulate scan parameters for two-dimensional medical images, such as x-rays. In some instances, selecting and adjusting the parameter set is applied to emulate different image capture settings such as portable chest x-rays taken laying down in the bed, sitting upright in a recliner or as a standing chest x-ray in a radiology department or facility. Each setting contributes to variations in alignment of the x-ray geometry and patient. Further, the orientation of the patient may cause shifts in the relative position of the various internal structures (e.g., organs or skeletal structures) which can also be modeled and used in the creation of the resulting images. Selection and adjustment of the parameter set may be used to provide a framework for creating simulated images of specified orientations, image capture settings, and other relevant and suitable aspects of medical images.

Where the second imaging type is x-ray data or other two-dimensional imaging, such as two-dimensional (e.g., planar) nuclear medicine imaging data, and the first imaging type is three-dimensional nuclear medicine imaging data, such as positron emission tomography scan data, embodiments described herein may generate simulated images of the second imaging type from the first imaging type. The second imaging type may also be nuclear medicine imaging data, such as single positron computed emission tomography (SPECT) or Fludeoxyglucose (18F-FDG) positron emission tomography (PET) scan data. In such embodiments, to generate synthetic images of the second imaging type (e.g., SPECT or PET) from the first imaging type (e.g., volumetric CT data), embodiments of the present disclosure may simulate a synthetic radiotracer distribution within a region of interest in the volumetric CT data. For example, 18F-FDG may be simulated as uniformly distributed within a bladder. Embodiments of the present disclosure may then simulate emission of photons from the region (e.g., photons traveling from the bladder, through tissue, to the PET detector) to obtain planar data (e.g., i.e., planar nuclear medicine projection data). These embodiments may then reconstruct three-dimensional PET/SPECT synthetic images from these projections. Although described with respect to specified imaging types, it should be understood that the first imaging type and the second imaging type may be any suitable or relevant medical imaging data, scan data, or imaging types.

In some embodiments, the modeling component 140 models the second medical imaging data using forward projection operations. The modeling component 140 may use forward projection analytical models, Monte-Carlo or statistical methods, combinations thereof, or any other suitable and relevant forward projection methods or operations. In some instances, the modeling component 140 combines forward projection operations with deep learning models to model the second medical imaging data. For example, in an x-ray imaging system, forward projection operations may model emissions of x-rays from an x-ray tube, through patient anatomy, to a detector. In nuclear medicine systems, the forward projection operations may model emission of gamma rays from a radioactive source, present within a patient, through the patient anatomy to a detector.

In some embodiments, modeling the second medical imaging data converts a first dimensionality of the first medical imaging data to a second dimensionality of the second medical imaging data. The first dimensionality may be three-dimensional, such that the first medical imaging data is a three-dimensional imaging data or scan. The second dimensionality may be two-dimensional, such that the second medical imaging data is a two-dimensional imaging data or scan. For example, where the first medical imaging data is CT scan data and the second medical imaging data is x-ray data, the modeling component 140 models x-ray data from the CT scan data. In some embodiments, modeling the second medical imaging data and converting dimensionality of the first medical imaging data, enables generation of tomosynthesis x-ray images from volumetric CT scan data. In such instances, the modeling component 140 may use linear tomography or tomosynthesis for converting dimensionality for generation of the tomosynthesis x-ray images. Although described with respect to specified methods of converting dimensionality, it should be understood that the modeling component 140 may convert the dimensionality of the first medical imaging data to the second medical imaging data using any suitable or relevant technique or combination of techniques.

In some embodiments, the generation component 110, the projection component 120, and the modification component 130 perform batch operations, automating generation of sets of synthetic images. The batched generation of synthetic images may be based on a set or corpus of source images (e.g., hundreds or thousands of images), parameter sets generated or selected for each batch, and automatically generated, as described in more detail below. Parameter sets, as discussed below, may enable multiple variations for each source image or source image set to generate one or more simulated output images. As discussed herein, the embodiments of the present disclosure may enable automatic or semi-automatic generation of any number of synthetic images. For example, the present disclosure may generate a single synthetic image, hundreds of synthetic images, or thousands of synthetic images with sets of synthetic images including one or more of the generated images.

At operation 240, the modeling component 140 generates an imaging model based on the set of modified projection images. The imaging model may be generated using one or more operations. In some embodiments, the imaging model is trained as a deep learning model using the set of modified projection images. The deep learning model may be trained using neural networking, convolutional neural networking, recurrent neural networking, deep convolutional networking, combinations thereof, or any other suitable neural networking methods. The modeling component 140 may then selectively tune the imaging model. In some embodiments, the modeling component 140 tunes the imaging model based on portions of the set of modified projection images. For example, where the set of modified projection images contain subsets of modified projection images for specific body parts or areas of a human body, the modeling component 140 may selectively tune the imaging model using particular body parts or areas. The modeling component 140 may also test the imaging model on one or more subsets of modified projection images. In such embodiments, once the imaging model is trained and tuned, the modeling component 140 may run the imaging model on specified modified projection images having known attributes. The known attributes may be specified abnormalities, indicators of disease, or other suitable attributes. The modeling component 140 may identify attributes or abnormalities within the specified modified projection images and compare the identified attributes or abnormalities with the known attributes of the specified modified projection images.

At operation 250, the access component 150 accesses a patient medical image. In some embodiments, the patient medical image is associated with a first imaging type. The patient medical images may be accessed by receiving the patient medical image at a computing system associated with the image simulation system 102. For example, the patient medical images may be received from a medical imaging device scanning or imaging one or more portions or areas of concern of a patient. The patient medical images may be received via a network connection or transferred from physical storage media. Once accessed, the access component 150 may pass the patient medical image to the identification component 160.

At operation 260, the identification component 160 identifies an attribute of interest on the patient medical image based on the imaging model. In some embodiments, the attribute of interest is an abnormality within a patient associated with the patient medical image. The identification component 160 may identify the attribute of interest by passing the patient medial image to the imaging model. The imaging model analyzes the patient medical image to determine the attribute of interest, such as abnormalities or indicators of disease, using characteristics or aspects of such attributes trained using the modified projection images. The imaging model may analyze the patient medical image using edge detection, object recognition, pattern recognition, semantic segmentation, shape recognition, line detection, combinations thereof, and other suitable image recognition operations.

In some embodiments, the identification component 160 identifies the attribute of interest by identifying an area of interest within the patient medical image. The area of interest indicates an area of the patient depicted in the patient medical image. The area of interest may be identified from one or more characteristics or aspects of the patient medical image. In some embodiments, the identification component 160 identifies the attribute of interest from metadata associated with the patient medical image. For example, the identification component 160 may identify an x-ray of an arm based on a label, one or more edges of the arm, a shape of the arm, an internal structure of the arm (e.g., a bone), or any other suitable identifying characteristics.

In some embodiments, the identification component 160 selects an area model based on the area of interest. The area model is an imaging model generated for a specified bodily area associated with the area of interest. The identification component 160 may select the area by matching the characteristics of the patient medical image, discussed above, with a label or indicator of a suitable area model. Using the area model, the identification component 160 identifies the attribute of interest on the patient medical image based on the area model. The identification component 160 may also select or use a portion of the imaging model, narrowing search or identification parameters of the imaging model based on the area of interest identified by the identification component 160. In such instances, the area model acts as a portion of the imaging model, without operating as a separate model.

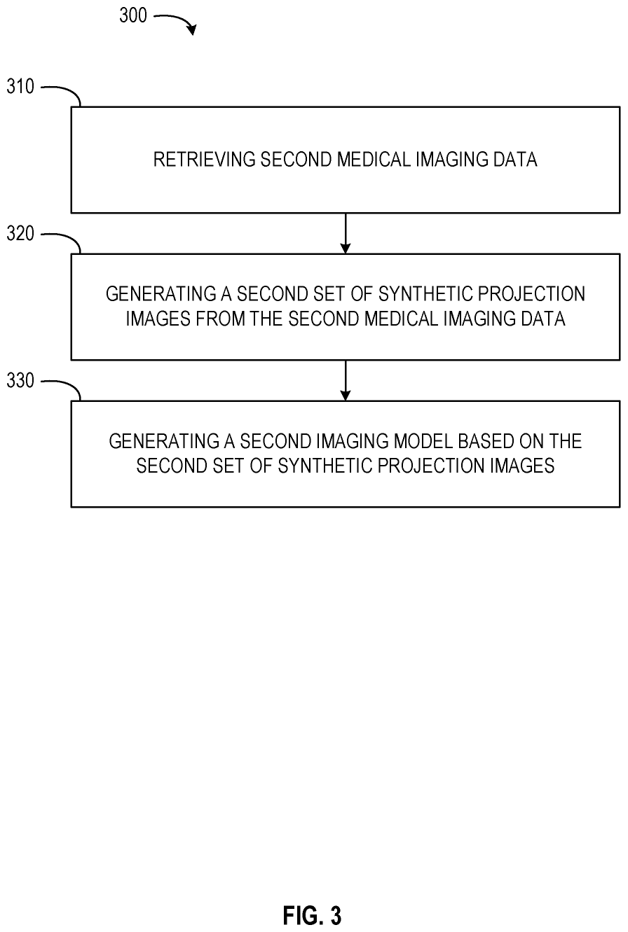

In some embodiments, the identification component 160 identifies the attribute of interest based on a plurality of imaging models. The identification component 160 may identify the attribute of interest using a plurality of area models. In some instances, the identification component 160 identifies the attribute of interest using one or more attribute models, trained or tuned for individual attributes or abnormalities of a specified area or portion of a patient. In some embodiments, the attribute of interest is identified based on the first imaging model and a second imaging model, described in more detail with respect to FIG. 3.

FIG. 3 shows a flow diagram of an embodiment of a computer-implemented method 300 for disease simulation and identification in medical images. The method 300 may be performed by or within the computing environment 100. In some embodiments, the method 300 comprises or incorporates one or more operations of the method 200. In some instances, operations of the method 300 may be incorporated as part of or sub-operations of the method 200.

In operation 310, the generation component 110 retrieves second medical imaging data. In some embodiments, the second medical imaging data is associated with a second imaging type. The second medical imaging data may be volumetric CT scan data. The generation component 110 may retrieve the second medical imaging data from an imaging repository containing actual medical images of the second imaging type. The imaging repository may also include synthetic imaging data, generated from one or more medical imaging data using modeling, projection, or image editing techniques, as discussed in the present disclosure. The imaging repository may include both real and synthetic medical imaging data.

In operation 320, the generation component 110 generates a second set of synthetic projection images from the second medical imaging data. The second set of synthetic projection images may be associated with the second imaging type. The second set of synthetic images may be generated from one or more actual medical images captured from patients. The generation component 110 may generate the second set of synthetic projection images in a manner similar to or the same as described above. In some embodiments, the generation component 110 generates the second set of synthetic projection images in batches. The batches may correspond to sets of synthetic projection images. A portion of the sets of synthetic projection images may include simulated diseases, tissue damage, imaging abnormalities or anomalies, tissue absorption issues, tissue density issues, or any other suitable or relevant irregularities. In some embodiments, after simulating suitable irregularities in at least a portion of the second medical imaging data, the generation component 110 generates the second set of synthetic projection images as a set or batch of medical images of the second imaging type. In some instances, after simulating suitable irregularities in at least a portion of the second medical imaging data, the generation component 110 may project the second medical imaging data of the second imaging type (e.g., volumetric CT data) into a synthetic image of the first imaging type (e.g., x-ray data). In some embodiments, the generation component 110 performs one or more operations to improve or adjust the generated second set of synthetic projection images.

The generation component 110 may subsequently generate a first set of synthetic projection images from the first medical imaging data, as described above in operation 210. The generation component 110 may also generate the first set of synthetic projection images prior to generating the second set of synthetic projection images.

In operation 330, the modeling component 140 generates a second imaging model based on the second set of synthetic projection images. The second set of synthetic projection images may be used to train deep learning algorithms related to the second imaging type. For example, where the second set of synthetic projection images are associated with volumetric CT scan data as the second imaging type, the second set of synthetic projection images may be used to train a deep learning algorithm, the second imaging model, related to radiation-based medical imaging and two-dimensional or three-dimensional nuclear medicine images. The radiation-based medical imaging may be a two-dimensional x-ray image. The nuclear medicine image may be a position emission tomography (PET) scan. In some embodiments, the second medical imaging data is projected onto the first set of synthetic projection images based on the second imaging model.

The second imaging model may also be generated based on the second set of synthetic projection images as a deep learning algorithm for analysis of medical images of the first imaging type. In such embodiments, the modeling component 140 generates the second imaging model from the second set of synthetic projection images which have been converted from the second image type to the first image type, as discussed above. For example, where volumetric CT data has been projected, as a set of synthetic projection images, into x-ray data, the second imaging model may be a second imaging model configured for analysis of x-ray images.

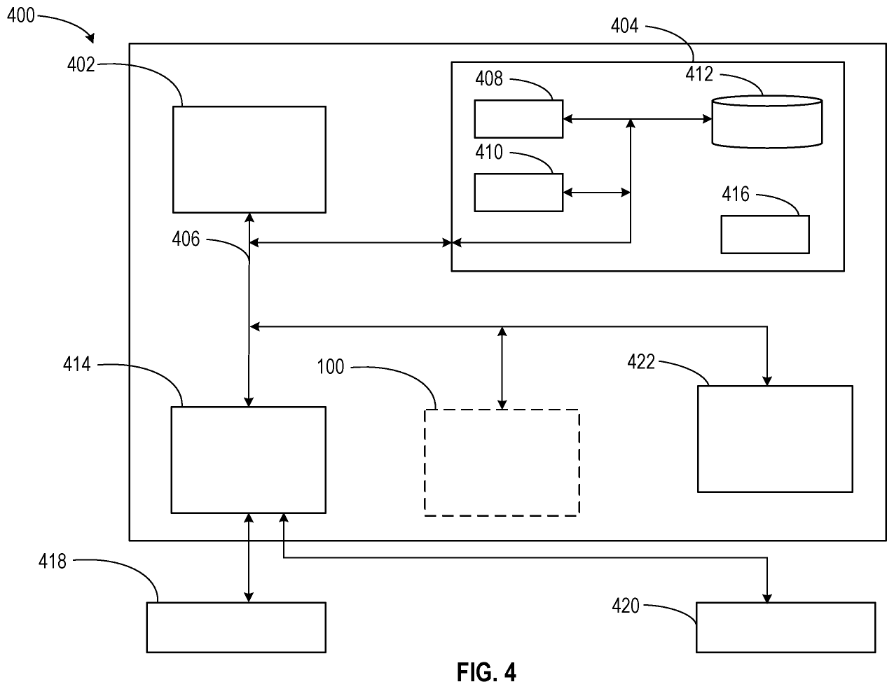

Embodiments of the present disclosure may be implemented together with virtually any type of computer, regardless of the platform being suitable for storing and/or executing program code. FIG. 4 shows, as an example, a computing system 400 (e.g., cloud computing system) suitable for executing program code related to the methods disclosed herein and for disease simulation and identification in medical images.

The computing system 400 is only one example of a suitable computer system and is not intended to suggest any limitation as to the scope of use or functionality of embodiments of the present disclosure described herein, regardless, whether the computer system 400 is capable of being implemented and/or performing any of the functionality set forth hereinabove. In the computer system 400, there are components, which are operational with numerous other general purpose or special purpose computing system environments or configurations. Examples of well-known computing systems, environments, and/or configurations that may be suitable for use with computer system/server 400 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like. Computer system/server 400 may be described in the general context of computer system-executable instructions, such as program modules, being executed by a computer system 400. Generally, program modules may include routines, programs, objects, components, logic, data structures, and so on that perform particular tasks or implement particular abstract data types. Computer system/server 400 may be practiced in distributed cloud computing environments where tasks are performed by remote processing devices that are linked through a communications network. In a distributed cloud computing environment, program modules may be located in both, local and remote computer system storage media, including memory storage devices.

As shown in the figure, computer system/server 400 is shown in the form of a general-purpose computing device. The components of computer system/server 400 may include, but are not limited to, one or more processors 402 (e.g., processing units), a system memory 404 (e.g., a computer-readable storage medium coupled to the one or more processors), and a bus 406 that couple various system components including system memory 404 to the processor 402. Bus 406 represents one or more of any of several types of bus structures, including a memory bus or memory controller, a peripheral bus, an accelerated graphics port, and a processor or local bus using any of a variety of bus architectures. By way of example, and not limiting, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus. Computer system/server 400 typically includes a variety of computer system readable media. Such media may be any available media that is accessible by computer system/server 400, and it includes both, volatile and non-volatile media, removable and non-removable media.

The system memory 404 may include computer system readable media in the form of volatile memory, such as random-access memory (RAM) 408 and/or cache memory 410. Computer system/server 400 may further include other removable/non-removable, volatile/non-volatile computer system storage media. By way of example only, a storage system 412 may be provided for reading from and writing to a non-removable, non-volatile magnetic media (not shown and typically called a ‘hard drive’). Although not shown, a magnetic disk drive for reading from and writing to a removable, non-volatile magnetic disk (e.g., a ‘floppy disk’), and an optical disk drive for reading from or writing to a removable, non-volatile optical disk such as a CD-ROM, DVD-ROM or other optical media may be provided. In such instances, each can be connected to bus 406 by one or more data media interfaces. As will be further depicted and described below, the system memory 404 may include at least one program product having a set (e.g., at least one) of program modules that are configured to carry out the functions of embodiments of the present disclosure.

The program/utility, having a set (at least one) of program modules 416, may be stored in the system memory 404 by way of example, and not limiting, as well as an operating system, one or more application programs, other program modules, and program data. Program modules may include one or more of the generation component 110, the projection component 120, the modification component 130, the modeling component 140, the access component 150, and the identification component 160, which are illustrated in FIG. 1. Each of the operating systems, one or more application programs, other program modules, and program data or some combination thereof, may include an implementation of a networking environment. Program modules 416 generally carry out the functions and/or methodologies of embodiments of the present disclosure, as described herein.

The computer system/server 400 may also communicate with one or more external devices 418 such as a keyboard, a pointing device, a display 420, etc.; one or more devices that enable a user to interact with computer system/server 400; and/or any devices (e.g., network card, modem, etc.) that enable computer system/server 400 to communicate with one or more other computing devices. Such communication can occur via Input/Output (I/O) interfaces 414. Still yet, computer system/server 400 may communicate with one or more networks such as a local area network (LAN), a general wide area network (WAN), and/or a public network (e.g., the Internet) via network adapter 422. As depicted, network adapter 422 may communicate with the other components of computer system/server 400 via bus 406. It should be understood that, although not shown, other hardware and/or software components could be used in conjunction with computer system/server 400. Examples include, but are not limited to: microcode, device drivers, redundant processing units, external disk drive arrays, RAID systems, tape drives, and data archival storage systems, etc.

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Service models may include software as a service (SaaS), platform as a service (PaaS), and infrastructure as a service (IaaS). In SaaS, the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings. In PaaS, the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations. In IaaS, the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment models may include private cloud, community cloud, public cloud, and hybrid cloud. In private cloud, the cloud infrastructure is operated solely for an organization. It may be managed by the organization or a third party and may exist on-premises or off-premises. In community cloud, the cloud infrastructure is shared by several organizations and supports specific community that has shared concerns (e.g., mission, security requirements, policy, and compliance considerations). It may be managed by the organizations or a third party that may exist on-premises or off-premises. In public cloud, the cloud infrastructure is made available to the general public or a large industry group and is owned by an organization selling cloud services. In hybrid cloud, the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling modularity, and semantic interoperability. At the heart of cloud computing is an infrastructure that includes a network of interconnected nodes.

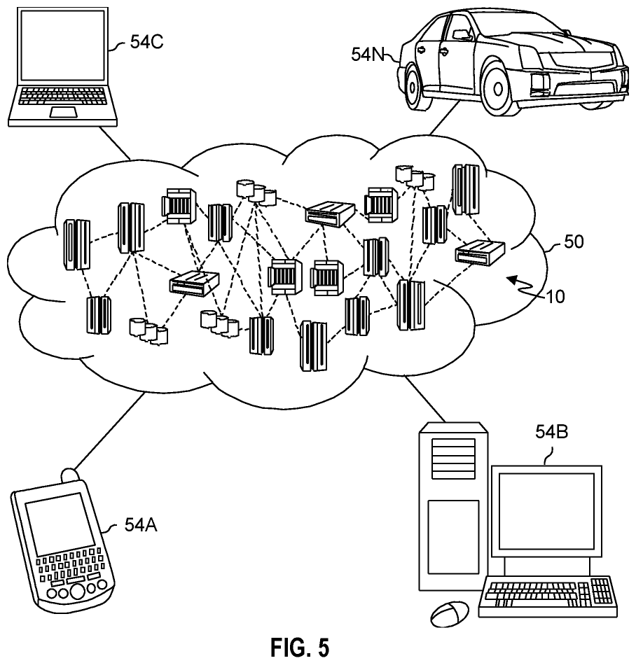

Referring now to FIG. 5, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

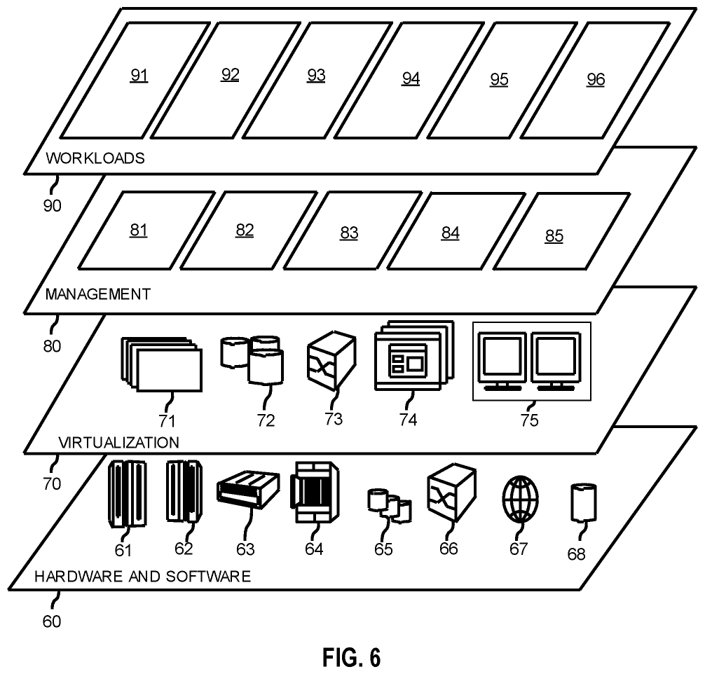

Referring now to FIG. 6, a set of functional abstraction layers provided by cloud computing environment 50 (FIG. 5) is shown. It should be understood in advance that the components, layers, and functions shown in FIG. 5 are intended to be illustrative only and embodiments of the disclosure are not limited thereto. As depicted, the following layers and corresponding functions are provided:

Hardware and software layer 60 includes hardware and software components. Examples of hardware components include: mainframes 61; RISC (Reduced Instruction Set Computer) architecture-based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and network traffic direction processing 96.

Cloud models may include characteristics including on-demand self-service, broad network access, resource pooling, rapid elasticity, and measured service. In on-demand self-service a cloud consumer may unilaterally provision computing capabilities such as server time and network storage, as needed automatically without requiring human interaction with the service's provider. In broad network access, capabilities are available over a network and accessed through standard mechanisms that promote use by heterogeneous thin or thick client platforms (e.g., mobile phones, laptops, and PDAs). In resource pooling, the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter). In rapid elasticity, capabilities can be rapidly and elastically provisioned, in some cases automatically, to quickly scale out and rapidly released to quickly scale in. To the consumer, the capabilities available for provisioning often appear to be unlimited and can be purchased in any quantity at any time. In measured service, cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

The descriptions of the various embodiments of the present disclosure have been presented for purposes of illustration, but are not intended to be exhaustive or limited to the embodiments disclosed. Many modifications and variations will be apparent to those of ordinary skills in the art without departing from the scope and spirit of the described embodiments. The terminology used herein was chosen to best explain the principles of the embodiments, the practical application or technical improvement over technologies found in the marketplace, or to enable others of ordinary skills in the art to understand the embodiments disclosed herein.

The present invention may be embodied as a system, a method, and/or a computer program product. The computer program product may include a computer-readable storage medium (or media) having computer readable program instructions thereon for causing a processor to carry out aspects of the present invention.

The computer-readable storage medium may be an electronic, magnetic, optical, electromagnetic, infrared or a semi-conductor system for a propagation medium. Examples of a computer-readable medium may include a semi-conductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), DVD and Blu-Ray-Disk.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disk read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer-readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages. The computer readable program instructions may execute entirely on the user's computer, partly on the user's computer as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatuses, or another device to cause a series of operational steps to be performed on the computer, other programmable apparatus or other device to produce a computer implemented process, such that the instructions which execute on the computer, other programmable apparatuses, or another device implement the functions/acts specified in the flowchart and/or block diagram block or blocks.

The flowcharts and/or block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or act or carry out combinations of special purpose hardware and computer instructions.

The terminology used herein is for the purpose of describing particular embodiments only and is not intended to limit the present disclosure. As used herein, the singular forms “a”, “an” and “the” are intended to include the plural forms as well, unless the context clearly indicates otherwise. It will further be understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or steps plus function elements in the claims below are intended to include any structure, material, or act for performing the function in combination with other claimed elements, as specifically claimed. The description of the present disclosure has been presented for purposes of illustration and description, but is not intended to be exhaustive or limited to the present disclosure in the form disclosed. Many modifications and variations will be apparent to those of ordinary skills in the art without departing from the scope of the present disclosure. The embodiments are chosen and described in order to explain the principles of the present disclosure and the practical application, and to enable others of ordinary skills in the art to understand the present disclosure for various embodiments with various modifications, as are suited to the particular use contemplated.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

What is claimed is:

1. A computer-implemented method, comprising:
generating a set of synthetic projection images from first medical imaging data of a first imaging type;
projecting second medical imaging data of a second imaging type onto the set of synthetic projection images;
modifying one or more synthetic projection images of the set of synthetic projection images to generate a set of modified projection images based on the second medical imaging data;
generating an imaging model based on the set of modified projection images;
obtaining a patient medical image of the first imaging type; and
identifying an attribute of interest on the patient medical image based on the imaging model.

2. The computer-implemented method of claim 1, wherein the set of synthetic projection images is a first set of synthetic projection images, the method further comprising:
retrieving second medical imaging data of the second imaging type; and
generating a second set of synthetic projection images from the second medical imaging data, the second set of synthetic projection images having the second imaging type.

3. The computer-implemented method of claim 2, wherein the imaging model is a first imaging model, the method further comprising:
generating a second imaging model based on the second set of synthetic projection images.

4. The computer-implemented method of claim 3, wherein the second medical imaging data is projected onto the first set of synthetic projection images based on the second imaging model.

5. The computer-implemented method of claim 3, wherein the attribute of interest on the patient medical image is identified based on the first imaging model and the second imaging model.

6. The computer-implemented method of claim 1, further comprising:
augmenting the set of synthetic projection images with a set of image tracking points.

7. The computer-implemented method of claim 1, further comprising:
identifying an area of interest within the patient medical image, the area of interest indicating an area of the patient depicted in the patient medical image; and
selecting an area model based on the area of interest, the area model being an imaging model generated for a specified bodily area associated with the area of interest.

8. The computer-implemented method of claim 1, wherein the attribute of interest is an abnormality within a patient associated with the patient medical image.

9. A system, comprising:
one or more processors; and
a computer-readable storage medium, coupled to the one or more processors, storing program instructions that, when executed by the one or more processors, cause the one or more processors to perform operations comprising:
generating a set of synthetic projection images from first medical imaging data of a first imaging type;
projecting second medical imaging data of a second imaging type onto the set of synthetic projection images;
modifying one or more synthetic projection images of the set of synthetic projection images to generate a set of modified projection images based on the second medical imaging data;
generating an imaging model based on the set of modified projection images;
obtaining a patient medical image of the first imaging type; and
identifying an attribute of interest on the patient medical image based on the imaging model.

10. The system of claim 9, wherein the set of synthetic projection images is a first set of synthetic projection images, the operations further comprising:
retrieving second medical imaging data of the second imaging type; and
generating a second set of synthetic projection images from the second medical imaging data, the second set of synthetic projection images having the second imaging type.

11. The system of claim 10, wherein the imaging model is a first imaging model, the operations further comprising:
generating a second imaging model based on the second set of synthetic projection images.

12. The system of claim 11, wherein the second medical imaging data is projected onto the first set of synthetic projection images based on the second imaging model.

13. The system of claim 11, wherein the attribute of interest of the patient medical image is identified based on the first imaging model and the second imaging model.

14. The system of claim 9, wherein the operations further comprise:
identifying an area of interest within the patient medical image, the area of interest indicating an area of the patient depicted in the patient medical image; and
selecting an area model based on the area of interest, the area model being an imaging model generated for a specified bodily area associated with the area of interest.

15. A computer program product comprising a computer readable storage medium having program instructions embodied therewith, the program instructions being executable by one or more processors to cause the one or more processors to perform operations comprising:
generating a set of synthetic projection images from first medical imaging data of a first imaging type;
projecting second medical imaging data of a second imaging type onto the set of synthetic projection images;
modifying one or more synthetic projection images of the set of synthetic projection images to generate a set of modified projection images based on the second medical imaging data;
generating an imaging model based on the set of modified projection images;
obtaining a patient medical image of the first imaging type; and
identifying an attribute of interest on the patient medical image based on the imaging model.

16. The computer program product of claim 15, wherein the set of synthetic projection images is a first set of synthetic projection images, the operations further comprising:
retrieving second medical imaging data of the second imaging type; and
generating a second set of synthetic projection images from the second medical imaging data, the second set of synthetic projection images having the second imaging type.

17. The computer program product of claim 16, wherein the imaging model is a first imaging model, the operations further comprising:
generating a second imaging model based on the second set of synthetic projection images.

18. The computer program product of claim 17, wherein the second medical imaging data is projected onto the first set of synthetic projection images based on the second imaging model.

19. The computer program product of claim 17, wherein the attribute of interest of the patient medical image is identified based on the first imaging model and the second imaging model.

20. The computer program product of claim 15, wherein the operations further comprise:
identifying an area of interest within the patient medical image, the area of interest indicating an area of the patient depicted in the patient medical image; and
selecting an area model based on the area of interest, the area model being an imaging model generated for a specified bodily area associated with the area of interest. P201901848US01 Page 31 of 32
